(19) United States
(12) Patent Application Publication
(10) Pub. No.: US 2024/0139153 A1
(43) Pub. Date: May 2, 2024

(54) Inhibition of Endothelial ETS Family Transcription Factors Promotes Flow-Dependent Ocular Vessel Regression

(71) Applicant: Oklahoma Medical Research Foundation, Oklahoma City, OK (US)

(72) Inventors: Christopher Schafer, Oklahoma City, OK (US); Courtney Griffin, Oklahoma City, OK (US)

(21) Appl. No.: 18/044,149

(22) PCT Filed: Sep. 15, 2021
(86) PCT No.: PCT/US2021/050491

§ 371 Date: Mar. 6, 2023

Related U.S. Application Data
(60) Provisional application No. 63/079,904, filed on Sep. 17, 2020.
(60) Provisional application No. 63/109,932, filed on Nov. 5, 2020.

(51) Int. Cl. A61K 31/404

(52) U.S. Cl. CPC A61K 31/404

(57) ABSTRACT

The present invention includes a method of inducing vascular regression in poorly perfused blood vessels in a subject comprising providing the subject with an effective amount of an inhibitor of an Endothelial ETS Family Transcription Factor. The compounds of the present invention are used in the treatment of retinopathy of prematurity (ROP), diabetic retinopathy (DR), or vascular malformations.

### CROSS-REFERENCE TO RELATED APPLICATIONS

This PCT International Application claims benefit to U.S. Provisional Application No. 63/079,904 filed on Sep. 17, 2020 and U.S. Provisional Application No. 63/109,932 filed on Nov. 5, 2020, the contents of which are incorporated by reference in their entirety.

### STATEMENT OF FEDERALLY FUNDED RESEARCH

This invention was made with government support under 1R35HL144605-01 awarded by the National Institutes of Health. The government has certain rights in the invention.

### TECHNICAL FIELD OF THE INVENTION

The present invention relates in general to the field of inducing vascular regression in poorly perfused blood vessels.

### INCORPORATION-BY-REFERENCE OF MATERIALS FILED ON COMPACT DISC

None.

### BACKGROUND OF THE INVENTION

Without limiting the scope of the invention, its background is described in connection with poorly perfused blood vessels.

Ocular blood vessels are regulated to maintain to balance the high nutritional demands of the retina against the impairment of visual function that results from hypervascularization¹. In diseases such as retinopathy of prematurity (ROP) and diabetic retinopathy (DR), this balance is lost and results in the formation of neovascular (NV) tufts originating from the superficial retinal vascular layer, which physically impede the sensation of light²⁻⁴. Moreover, retinal neovessels are inherently unstable and prone to hemorrhage, which then elevates ocular inflammation and further exacerbates visual dysfunction^{5,6}. Because of this, ROP and DR are among the leading causes of visual dysfunction in infants and adults, respectively^{7,8}.

For decades, the ablation of the peripheral retina by laser-based photocoagulation or cryotherapy has been used to curtail the progression of NV disorders⁹⁻¹¹. However, these treatments fail to reverse visual defects acquired prior to the onset of treatment and are associated with the loss of peripheral and night vision^{12,13}. Such limitations have motivated the development of therapies aimed at the inhibition of pro-angiogenic vascular endothelial growth factor (VEGF) signaling to reduce the extent of vascular overgrowth¹⁴⁻¹⁷. VEGF has long been recognized as an important pro-angiogenic signaling molecule, and it is well established that VEGF plays a role in the progression of NV disease^{18,19}. In a randomized study, intravitreal injection of bevacizumab, a monoclonal VEGF-A antibody, was more effective than conventional laser-based therapy for treating ROP¹⁶. However, VEGF plays an essential role in many developmental processes, raising concerns about long-term consequences of its inhibition, particularly in infants with ROP. For example, one study demonstrated a reduction in systemic VEGF for 2 months after an intravitreal anti-VEGF treatment²⁰. Moreover, in longitudinal studies anti-VEGF treatments have shown a tendency for reactivation of NV complications after treatment is suspended^{21,22} as well as apparent long-term complications in retinal vascular structure and ocular function^{23,24}.

Therefore, a need remains for novel treatment for retinopathy of prematurity (ROP) and/or diabetic retinopathy (DR) that are effective and that do not conflict with developmental processes.

### SUMMARY OF THE INVENTION

In one embodiment, the present invention includes a method of inducing vascular regression in poorly perfused blood vessels in a subject comprising providing the subject with an effective amount of an inhibitor of an Endothelial ETS Family Transcription Factor. In one aspect, the subject is in need of treatment for retinopathy of prematurity (ROP), diabetic retinopathy (DR), or vascular malformations. In another aspect, the inhibitor of an Endothelial ETS Family Transcription Factor is selected from an siRNA, RNAi, an RNAse inhibitor, or a small molecule inhibitor. In another aspect, the inhibitor of an Endothelial ETS Family Transcription Factor is an RNA Helicase A inhibitor. In another aspect, the inhibitor of an Endothelial ETS Family Transcription Factor is YK 4-279 or TK216 having the formula:

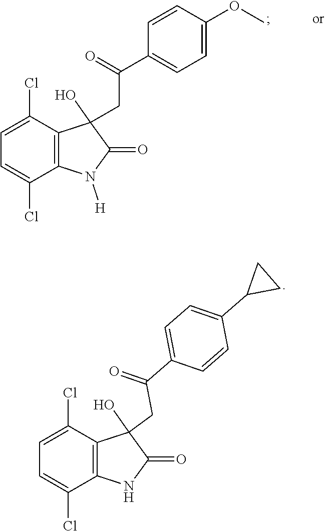 [SMILES: COc1ccc(C(=O)CC2(O)C(=O)Nc3c(Cl)ccc(Cl)c32)cc1 ; O=C(CC1(O)C(=O)Nc2c(Cl)ccc(Cl)c21)c1ccc(C2CC2)cc1]

In another aspect, the molecule has the formula:

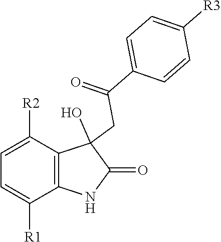

wherein, R1, R2, R3 are the same or different and are each independently hydrogen, halogen, Cl, Br, F, I, OH, a saturated or unsaturated alkyl group, a cycloalkyl group, a substituted or unsubstituted aryl group, an alkoxy group, an aryl group, a nitrone group or a selected from R′COO, R′COOCH(R″), R′CH═CH, R′CONH, R′CONH(R″); wherein R′, R″ are each independently a saturated or unsaturated alkyl, ring, an alkyl group, a substituted or unsubstituted aryl group, or an aromatic hetero group.

In another aspect, the method further comprises measuring vascular regression in poorly perfused blood vessels by hyaloid regression. In another aspect, the inhibitor of an Endothelial ETS Family Transcription Factor is administered topically, subconjunctivally, intracamerally, subtenonally, subretinally, subchoroidally, suprachoroidally, supraorbitally, retrobulbarlly, as an ocular implant, or intravitreally, and wherein the composition is an eye drop, gel, ointment, spray, a reservoir, or mist. In another aspect, the inhibitor of an Endothelial ETS Family Transcription Factor at least one of: decrease retinal neovessels or vascular malformations by at least 40% or a retinal avascular area by at least 60% compared to a vehicle-injected contralateral eye. In another aspect, the inhibitor of an Endothelial ETS Family Transcription Factor does not inhibit vascular endothelial growth factor (VEGF).

In another embodiment, the present invention includes a method of inducing vascular regression in poorly perfused blood vessels comprising: identifying a subject in need of treatment for neovascularization; and providing the subject with an effective amount of an inhibitor of an Endothelial ETS Family Transcription Factor. In one aspect, the subject is in need of treatment for retinopathy of prematurity (ROP), diabetic retinopathy (DR), or vascular malformations. In another aspect, the inhibitor of an Endothelial ETS Family Transcription Factor is selected from an siRNA, RNAi, an RNAse inhibitor, or a small molecule inhibitor. In another aspect, the inhibitor of an Endothelial ETS Family Transcription Factor is an RNA Helicase A inhibitor. In another aspect, the inhibitor of an Endothelial ETS Family Transcription Factor is YK 4-279 or TK216 having the formula:

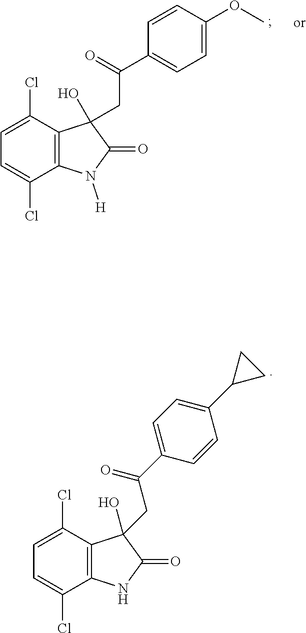 [SMILES: COc1ccc(C(=O)CC2(O)C(=O)Nc3c(Cl)ccc(Cl)c32)cc1 ; O=C(CC1(O)C(=O)Nc2c(Cl)ccc(Cl)c21)c1ccc(C2CC2)cc1]

In another aspect, the molecule has the formula:

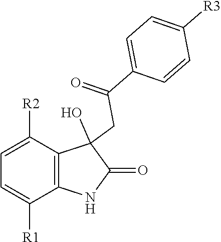

wherein, R1, R2, R3 are the same or different and are each independently hydrogen, halogen, Cl, Br, F, I, OH, a saturated or unsaturated alkyl group, a cycloalkyl group, a substituted or unsubstituted aryl group, an alkoxy group, an aryl group, a nitrone group or a selected from R′COO, R′COOCH(R″), R′CH═CH, R′CONH, R′CONH(R″); wherein R′, R″ are each independently a saturated or unsaturated alkyl, ring, an alkyl group, a substituted or unsubstituted aryl group, or an aromatic hetero group.

In another aspect, the method further comprises measuring vascular regression in poorly perfused blood vessels by hyaloid regression. In another aspect, the inhibitor of an Endothelial ETS Family Transcription Factor is administered topically, subconjunctivally, intracamerally, subtenonally, subretinally, subchoroidally, suprachoroidally, supraorbitally, retrobulbarlly, as an ocular implant, or intravitreally, and wherein the composition is an eye drop, gel, ointment, spray, a reservoir, or mist. In another aspect, the inhibitor of an Endothelial ETS Family Transcription Factor at least one of: decrease retinal neovessels or vascular malformations by at least 40% or a retinal avascular area by at least 60% compared to a vehicle-injected contralateral eye. In another aspect, the inhibitor of an Endothelial ETS Family Transcription Factor does not inhibit vascular endothelial growth factor (VEGF).

In another embodiment, the present invention includes a method for treating a retinopathy of prematurity (ROP), diabetic retinopathy (DR), or vascular malformation patient with inhibitor of an Endothelial ETS Family Transcription Factor, the method comprising the steps of: performing or having performed a vascular regression analysis in a poorly perfused blood vessel; and if the patient has vascular regression then treating the patient with an inhibitor of an Endothelial ETS Family Transcription Factor, wherein there is a decrease in retinal neovessels or vascular malformations, a decrease in a retinal avascular area, or both a vehicle-injected contralateral eye. In one aspect, the inhibitor of an Endothelial ETS Family Transcription Factor is YK 4-279 or TK216 having the formula:

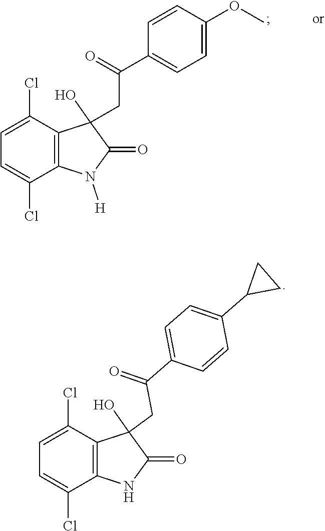 [SMILES: COc1ccc(C(=O)CC2(O)C(=O)Nc3c(Cl)ccc(Cl)c32)cc1 ; O=C(CC1(O)C(=O)Nc2c(Cl)ccc(Cl)c21)c1ccc(C2CC2)cc1]

In another aspect, the molecule has the formula:

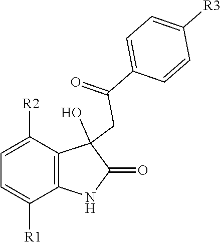

wherein, R1, R2, R3 are the same or different and are each independently hydrogen, halogen, Cl, Br, F, I, OH, a saturated or unsaturated alkyl group, a cycloalkyl group, a substituted or unsubstituted aryl group, an aryl group, an alkoxy group, a nitrone group or a selected from R′COO, R′COOCH(R″), R′CH═CH, R′CONH, R′CONH(R″); wherein R′, R″ are each independently a saturated or unsaturated alkyl, ring, an alkyl group, a substituted or unsubstituted aryl group, or an aromatic hetero group.

In another embodiment, the present invention includes a method of inducing vascular regression in poorly perfused blood vessels in a subject comprising providing the subject with an effective amount of an inhibitor that blocks the interaction between one or more ETS factors and one or more Krüppel-like factor (KLF) proteins. In one aspect, the subject is in need of treatment for retinopathy of prematurity (ROP), diabetic retinopathy (DR), or vascular malformations. In another aspect, the method further comprises measuring vascular regression in poorly perfused blood vessels by hyaloid regression. In another aspect, the inhibitor is administered topically, subconjunctivally, intracamerally, subtenonally, subretinally, subchoroidally, suprachoroidally, supraorbitally, retrobulbarlly, as an ocular implant, or intravitreally, and wherein the composition is an eye drop, gel, ointment, spray, a reservoir, or mist. In another aspect, the inhibitor at least one of: decrease retinal neovessels or vascular malformations by at least 40% or a retinal avascular area by at least 60% compared to a vehicle-injected contralateral eye. In another aspect, the inhibitor does not inhibit vascular endothelial growth factor (VEGF).

### BRIEF DESCRIPTION OF THE DRAWINGS

For a more complete understanding of the features and advantages of the present invention, reference is now made to the detailed description of the invention along with the accompanying figures and in which:

FIGS. 1A and 1B shows: Visualization and quantification of hyaloid vessel regression. (FIG. 1A) Hyaloid vessels from P4 and P8 mice were dissected and visualized by flat mount imaging with Isolectin-B4 (green). Scale bar=500 μm (FIG. 1B) Hyaloid vessel regression was quantified by counting the number of vessels crossing a line drawn at 50% of the total hyaloid diameter (dotted line in A). *P<0.05 (two-tailed Student t test).

FIGS. 2A to 2E show: Segmental apoptosis in constricted hyaloid vessels (FIG. 2A) Hyaloid vessel flat mount from a wild type P8 mouse visualized for TUNEL (green) and CD31 (red). Inset, magnified view of boxed region demonstrating the coordination of apoptosis to distinct vascular branches. Scale bar=500 μm. (FIG. 2B) Quantification of TUNEL⁺ vessel length as a percent of total hyaloid vessel length in P4 and P8 wild type mice. (FIG. 2C) Quantification of vessel diameters for non-apoptotic and apoptotic vessels from P4 and P8 wild type mice. (FIG. 2D) Hyaloid vessels from a P6 wild type mouse were immunostained for ECs (Isolectin-B4; green) and RBCs (Ter119; red) to visualize the exclusion of RBCs from constricted hyaloid vessels, white arrows. Scale bar=100 μm. *P<0.05 (two-tailed Student t-test). FIG. 2E Active caspase 3 immunostain in constricting hyaloid vessels. Immunostain of hyaloid vessels dissected from a P6 wild type mouse and visualized for Isolectin-B4 (green) and active caspase-3 (red). Active caspase-3 signal is confined to individual vascular branches which are constricted relative to adjacent vessel branches that have not initiated vessel death as evidenced by the lack of active caspase-3 signal. Scale bar=100 μm.

FIGS. 3A to 3D show: The transcription factor ERG is downregulated in constricted hyaloid vessels (FIG. 3A) Flat mount image of hyaloid vessels from a P6 wild type mouse visualized with Isolectin-B4 (red) and ERG (white). Arrows indicate a constricted hyaloid vessel with reduced nuclear ERG expression compared with an adjacent vessel. Scale bar=100 μm. (FIG. 3B) Immunostain of hyaloid vessels from a P8 wild type mouse visualized for Isolectin-B4 (red), ERG (white), and TUNEL (green). Arrow in magnified inset demonstrates downregulation of ERG in a constricted hyaloid vessel that is not yet TUNEL⁺. Scale bar=100 μm. (FIG. 3C) Immunostain of P6 hyaloid vessels for Isolectin-B4 (red), ERG (white), and VE-Cadherin (green). Arrows indicate constricted hyaloid vessels with reduced expression of ERG and VE-Cadherin. Scale bar=50 μm. (FIG. 3D) Comparison of ERG expression in retinal versus hyaloid ECs (A) Cross section of an eye from a P8 wild type mouse allowing comparison of ERG (green) expression between retinal and hyaloid ECs visualized with Isolectin-B4 (red). Inset, visualizing of the ERG channel alone for hyaloid (top) and retinal (bottom) ECs demonstrating the absence of ERG expression in regressing, hyaloid ECs. Scale bar=50 μm.

FIGS. 4A to 4G show: Intravitreal injection of YK-4-279 induces hyaloid vessel regression (FIG. 4A) Isolectin-B4-stained (green) image of flat mounted from P7 wild type mice given intravitreal injections of YK-4-279 at P5. Scale bar=500 μm. (FIG. 4B) Quantification of regression in hyaloids treated with YK-4-279 as in (FIG. 4A). (FIG. 4C) Quantification of regression in P7 hyaloids from Erg^{iECko} mice and wild type littermates following oral administration of tamoxifen at P3, P4 and P5. *P<0.05 (two-tailed Student t-test). FIG. 4D. Expression of FLI1 in hyaloid vessels. (FIG. 4D) Immunostain for Isolectin-B4 (green) and ERG (magenta) using P7 hyaloid vessels from an ErgiECko and wild type littermate control. Oral administration of tamoxifen at P3, P4, and P5 results in a loss of ERG expression in hyaloid ECs. Scale bar=100 uM (FIG. 4E) Immunostain for CD31 (green) and FLI1 (red) in P7 hyaloids from a wild type mouse. As observed for ERG, FLI1 expression is lowered in a constricted hyaloid vessel. Interestingly, some ECs of the constricted vessel appear to express FLI1 (arrow). However, it no longer appears to be colocalized with the nuclear DAPI stain as seen in the adjacent perfused vessel. Scale bar=50 uM (FIG. 4F) Alignment of murine ERG (SEQ ID NO:1) and FLI1 (SEQ ID NO:2) expression for identical (black highlight) and similar (grey highlight) residues. The highly conserved ETS DNA binding domain is indicated in red. FIG. 4G it shows that YK-4-279 blocks binding of ERG and of RNA Polymerase II to the promoter of the Cdh5 gene (VE-Cadherin) thereby demonstrating the YK-4-279 blocks ETS factor-mediated transcription in endothelial cells.

FIGS. 5A to 5D show: YK-4-279 induces the regression of 3D HUVEC cultures in vitro. (FIG. 5A) 3D lumenized HUVEC cultures (see METHODS) were treated with the indicated concentrations of YK-4-279 for 48 h followed by assessment of EC luminal area by toluidine blue stain. (FIG. 5B) Quantification of average lumen area in 3D HUVEC cultures treated with YK-4-279 as in (A). *P<0.05 (two-tailed Student t-test). (FIG. 5C) Potentiation of YK-4-279-induced regression by inflammatory cytokines. Western blot of 3D HUVEC cultures treated with the indicated YK-4-279, TNFα, and IL1β concentrations for pro-caspase 3 and actin. Reduced pro-caspase 3 signal following YK-4-279 treatment indicates elevated EC apoptosis that is further increased by co-treatment with low concentrations of both TNFα and IL1β. (FIG. 5D) Quantification of 3D HUVEC lumen area under the indicated YK-4-279, TNFα, and IL1β concentrations. Co-incubation of TNFα or IL1β with YK-4-279 further increases the extent of vascular regression in vitro. *P<0.05 (versus Control, unpaired Student t-test), \hat{ }P<0.05 (versus YK-4-279 alone, unpaired Student t-test).

FIGS. 6A to 6C shows: YK-4-279 induces flow-dependent HUVEC apoptosis in vitro (FIG. 6A) Image of HUVECS stained for CD31 (green) and active caspase-3 (red) under the indicated flow and YK-4-279 treatment conditions White arrows indicate apoptotic cells staining positive for active caspase-3 following YK-4-279 treatment under static, but not flow (10 dyn/cm²) conditions. Scale bar=30 μm. (FIG. 6B) Quantification of cells/μm² using experimental conditions in (FIG. 6A). (FIG. 6C) Quantification of HUVEC morphology by measuring the EC axis parallel to flow relative to the axis perpendicular to flow. *P<0.05 (two-tailed Student t-test).

FIGS. 7A to 7C show: YK-4-279 reduces neovascularization and improves retinal vascular structure in mice following oxygen-induced retinopathy (FIG. 7A) Representative images of P20.5 retinas immunostained for CD31 (black). Shown are retinas from an individual mouse treated with YK-4-279 and a vehicle control in contralateral eyes by intravitreal injection at P18.5 following the OIR protocol. Retinal neovessels and avascular area are outlined in red and blue, respectively. Retinal neovascular area (FIG. 7B) and avascular area (FIG. 7C) were quantified and compared between YK-4-279-injected and vehicle-injected eyes. *P<0.05 (paired two-tailed Student t-test).

FIG. 8A to 8C show: YK-4-279 does not affect wild type healthy retinal vessels. (FIG. 8A) Flat mounts of retinal vasculature from adult wild type mice were immunostained for CD31 (white) 48 hr after intravitreal injection of YK-4-279 or a vehicle control. Vascular length (FIG. 8B; n=4) and branch points (FIG. 8C; n=4) from retinas treated as in (FIG. 8A) were quantified using AutoTube and normalized to retinal area per image. NS=not significant (two-tailed Student's t-test). Error bars=S.D.

FIGS. 9A to 9C are graphs that show the transcriptional downregulation of the ERG target genes Erg (autoregulation; 9A), Cdh5 (9B), and Thbd (9C) in Human Umbilical Vein Endothelial Cells (HUVECs) with the indicated inhibitors (5 μM) for 8 h.

FIGS. 10A to 10D show that human umbilical vein endothelial cells were treated with the indicated inhibitor concentrations for 24 hr prior to phase contrast imaging. Both STK068867 and AQ-911 treatment result in loss of cell density suggesting the promotion of cell death as observed for YK-4-279.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

The present inventors recognized that an important feature of NV therapeutics is the ability to eliminate retinal neovessels or vascular malformations that form prior to the onset of treatment^{25,26}. For example, it is intriguing that certain developmental ocular blood vessel networks naturally undergo regression²⁷⁻³⁰. The underlying mechanisms of the ocular blood vessel regression processes can used to design of a new class of treatments aimed at promoting the regression of vascular abnormalities in NV disease. One well-documented example of physiological vascular regression occurs with the hyaloid vessels, which extend from the optic nerve head and through the vitreous, wrapping around the lens to nourish the development of the anterior segment of the eye. Shortly after birth in mice (and at midgestation in humans), the hyaloid vessels initiate a regression process culminating in their complete elimination within 2-3 weeks²⁸. Failed execution of this process results in a condition called persistent hyperplastic vitreous, in which the remaining hyaloid vessels impair visual function similarly to retinal neovessels found in NV disorders³¹.

Hyaloid vessel regression is dependent on macrophages, which initiate regression via the production of Wnt7b that induces apoptosis of vascular endothelial cells (ECs)³²⁻³⁴. However, the broad expression of Wnt7b³⁵ and its pro-angiogenic function in other contexts³⁶ suggests that additional factors are necessary for the induction of hyaloid regression. Indeed, other factors that influence vascular regression include decreased blood flow³⁷, VEGF deprivation³⁸, Angiopoietin-2³⁹, and inflammatory cytokines⁴⁰. Therefore, hyaloid regression results from the integration of many external stimuli thereby preventing the improper execution of a costly and irreversible vascular fate decision.

Thus, the present inventors sought to understand and take advantage of the mechanism underlying hyaloid vessel regression to yield viable pro-regressive therapeutic targets. The inventors have identified a class of EC-specific transcription factors that are strikingly downregulated in regressing hyaloid vessels. Pharmacological inhibition of these proteins, which belong to the E-26 transformation-specific (ETS) family of transcription factors (TFs), resulted in the induction of vascular regression in vivo and in vitro. Moreover, the inventors found that inhibitor treatment significantly resolved vascular abnormalities in a murine oxygen-induced retinopathy (OIR) model of ROP, vascular malformations (such as venous malformations) characterized by slow/tortuous blood flow, demonstrating the therapeutic potential of targeting vascular ETS family TFs to promote regression of pathological ocular blood vessels. Generally, venous malformations grow superficially, thus, they may be treatable topically or with direct injection of drugs into the malformation site.

The present invention includes the use of YK-4-279, TK216, or derivatives thereof, to promote the regression of pathological neovessels that are a hallmark of prevalent ocular diseases such as retinopathy of prematurity and diabetic retinopathy, or vascular malformations. Current treatments for this disease (such as Bevacizumab) focus on the inhibition of the pro-angiogenic molecular VEGF. These treatments prevent further retinal vascularization as well as help remove neovessels or vascular malformations. However, VEGF signaling plays an essential role in development. Therefore, inhibition of VEGF signaling, particularly in infants with retinopathy of prematurity, will have unwanted side effects. The inventors demonstrate herein a new approach in which treatment of neovascular disease is targeted directly at molecular pathways that promote vascular regression. The novel therapeutic reduces retinal neovessels or vascular malformations without the unwanted effects of VEGF inhibition. The inhibitor of the Endothelial ETS Family Transcription Factor is formulated for, and can be administered topically, subconjunctivally, intracamerally, subtenonally, subretinally, subchoroidally, suprachoroidally, supraorbitally, retrobulbarlly, as an ocular implant, or intravitreally. In non-limiting examples, the composition is formulated as an eye drop, serum, gel, ointment, spray, in a reservoir, or mist.

One inhibitor of an Endothelial ETS Family Transcription Factor is YK 4-279 having the formula:

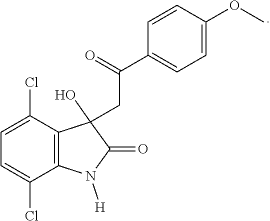 [SMILES: COc1ccc(C(=O)CC2(O)C(=O)Nc3c(Cl)ccc(Cl)c32)cc1]

Another inhibitor of an Endothelial ETS Family Transcription Factor is TK216 having the formula:

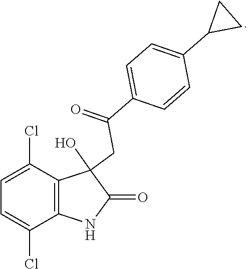 [SMILES: O=C(CC1(O)C(=O)Nc2c(Cl)ccc(Cl)c21)c1ccc(C2CC2)cc1]

The molecule may have the general formula:

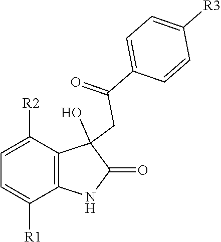

R1, R2, R3 are the same or different and are each independently hydrogen, a halogen, Cl, Br, F, I, OH, a saturated or unsaturated alkyl group (e.g., a C₁₋₆ alkyl), a cycloalkyl group, a substituted or unsubstituted aryl group, an aryl group, an alkoxy group (e.g., a C₁₋₆ alkoxy), a nitrone group or a selected from R′COO, R′COOCH(R″), R′CH═CH, R′CONH, R′CONH(R″); wherein R′, R″ are each independently a saturated or unsaturated alkyl, ring, an alkyl group, a substituted or unsubstituted aryl group, or an aromatic hetero group. Specific examples of the molecules tested are found hereinbelow.

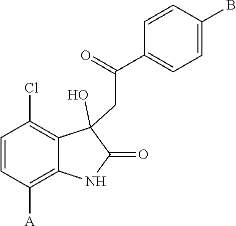

| | A | B |
|---|---|---|
| YK-4-279 | Cl | 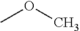 |
| T13166 (TK216) | Cl | 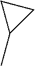 |
| STK068867 | CH₃ | 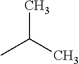 |
| AK-778 | CH₃ | 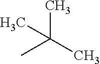 |
| AQ-911 | Cl | Cl |

Mice. C57Bl/6J (The Jackson Laboratory; #000664). Erg^{flox} (The Jackson Laboratory; #030988). Cdh5(PAC)-Cre^{ERT2} (Gift of Ralf Adams; currently available through Taconic: #13073)⁴¹. Mice were maintained and bred at the Oklahoma Medical Research Foundation (OMRF) animal facility. All protocols were approved by the OMRF Institutional Animal Care and Use Committee. Mice used in this study include wild type (C57Bl6J, JAX), Erg^{flox}, Cdh5(PAC)-CreERT2.

Hyaloid Dissection and Quantification. Hyaloid were dissected and quantified as described previously. Briefly, eyes were enucleated from P4-P8 mice and fixed for 30 min in 4% paraformaldehyde (PFA). The eyes were then transferred to PBS and dissected by removal of the lens and sclera leaving the retinal cup in which the hyaloids were loosely wrapped around the lens. The lens was then carefully removed from which the hyaloids were gently removed. The hyaloids were then transferred in a drop of PBS and flat mounted for imaging by carefully removing the remaining PBS.

Quantification of hyaloid regression was accomplished by drawing an outline of hyaloid flat mounts (labeled with Isolectin-B4) which was then decreased in size by 50% and centered over the hyaloid flat mount. Blind counts of hyaloid vessel number were performed by counting the number of vessels which crossed the 50% outline.

Immunofluorescence Imaging. Hyaloid flat mounts were dried for 30 min at room temperature then incubated overnight in 1% BSA, 0.5% Triton-X100 in PBS overnight at 4° C. Antibodies were diluted in 1% BSA in PBS and incubated overnight at 4° C. or 3 h at room temperature in the dark for primary and secondary antibody, respectively. Antibodies used in this study include ERG, CD31, Ter119, VE-Cadherin, and Active Casp3. Terminal deoxynucleotidyl transferase dUTP nick end labeling (TUNEL) staining was performed with the In Situ Cell Death Detection Kit, Fluorescein, purchased from Millipore Sigma (#11684795910), following the manufacturer's protocol.

Eye cross sections were prepared by collecting 10 μm thick cryosections from P8 wild type eyes embedded in OCT (Tissue-Tek). Sections were dried at room temperature for 30 min, washed with PBS, then blocked and permeabilized. All imaging was performed on either a Nikon Eclipse Ti-E or a Nikon C2 confocal with NIS-Elements software.

Intravitreal YK-4-279 Injections. Hyperthermia-induced anesthesia of wild type P5 mice was accomplished by submerging pups in ice for 5-8 min after placing them in a latex blanket wrapped in foil to avoid direct contact between the pup and ice. Once anesthetized (assessed by toe pinch) eyes were administered a drop of proparacaine hydrochloride ophthalmic solution (Akorn), followed by gentle exposure of the eye globe. Intravitreal injection of 70 nL of a 150 μmol/L YK-4-279 (Cayman Chemicals #13661 or AdooQ #A11612) solution (reaching a final concentration of ˜10 μmol/L in an estimated vitreal volume of ˜1 μL) or vehicle (0.9% sterile saline) was performed using a Nanoject II (Drummond) nanoinjector. Eyelids were then reclosed around the eye, and erythromycin ophthalmic ointment (Akorn) was applied. Pups were warmed by hand until they regained consciousness and returned to their cages for 2 d.

Vasculogenic 3D collagen assays. Human Umbilical Vein ECs were purchased from Lonza and used from passages 3 to 6 as previously described^{42,43}. ECs were suspended in 2.5 mg/mL collagen type I matrices, and assays were performed as described^{42,43}. With the exception that the culture media contained reduced serum supplement (RSII), ascorbic acid, FGF-2, Stem cell factor (SCF) at 40 ng/ml and interleukin-3 (IL-3) were added at 40 ng/ml. Stromal-derived factor-1α (SDF-1α) was added at 200 ng/ml into collagen type I matrices. Cultures were incubated at 37° C. in serum-free defined media and allowed to assemble over time. Cultures were fixed at 72 hr with 3% glutaraldehyde before staining with 0.1% toluidine blue in 30% methanol for nonfluorescent visualization. Lumen area was quantified by using Metamorph software as previously described^{42,43}. Individual data points were obtained from a triplicate wells and a minimum of 12 independent fields from these wells.

Bioassays with pharmacologic agents and cytokines. YK-4-279 (Cayman Chemicals #13661) was added to the cultures 48 hr after tube formation, at doses ranging from 20 μM to 0.15 μM. Proinflammatory Mediators, IL (Interleukin)-1β, TNF (Tumor Necrosis Factor) α, and Thrombin were added to cultures at different doses from 0 hr with or without YK-4-279. In some assays, triplicate wells were lysed with sample buffer and Western blots were performed to probe for pro-caspase 3 and actin.

Cell Culture/Flow Studies. Flow-based cell culture was performed using HUVECs (ATCC; #PCS-100-010) cultured in complete EGM-2 media (Lonza). HUVECs were cultured on Ibidi Luer^{0,6} flow slides and allowed to grow at 37° C. in 5% CO₂ for 24 h to a confluency of 80-90%. Slides were then attached to an Ibidi pump system with Perfusion Set Red (1.6 mm, #10962) and exposed to a sheer stress of 10 dyn/cm² for 24 h. Static conditions were achieved by similarly plating HUVECs in flow slides that were not exposed to flow. After 24 hr equilibration to flow conditions, cells were treated with 10 μmol/L YK-4-279 (or vehicle) for 24 hr. Cells were gently washed with 1 mL ice-cold PBS, then fixed and permeabilized by incubation with ice-cold methanol for 5 min. Fixed cells were treated with 1% BSA, 0.02% Triton X-100 for 1 hr at room temperature. Primary and secondary antibodies were added in 1% BSA and incubated for 2 hr at room temperature.

OIR Studies/Quantification. Oxygen Induced Retinopathy with wild type C57BL/6J mice was performed as previously described^{44,45}. Generally, pups with their dams were maintained in room air from birth until P7. From P7 to P12 pups were exposed to 75% O₂ in an oxygen chamber regulated by an OxyCycler Model A84. After 2.5 d of oxygen exposure, the dams were replaced with healthy lactating dams to prevent oxygen toxicity. At P12 pups were returned to room air until P18.

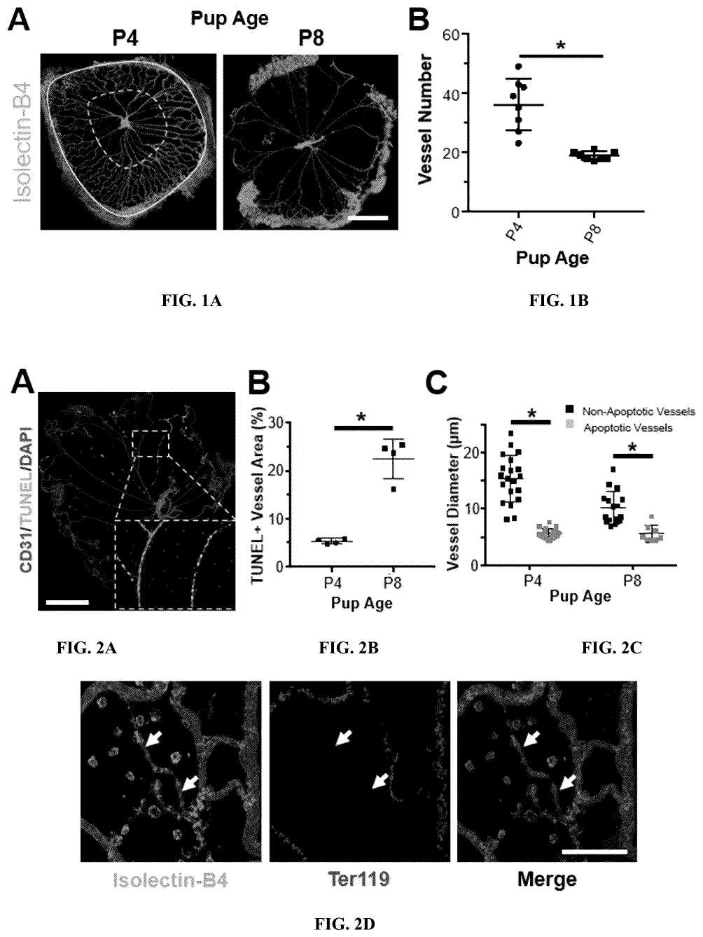

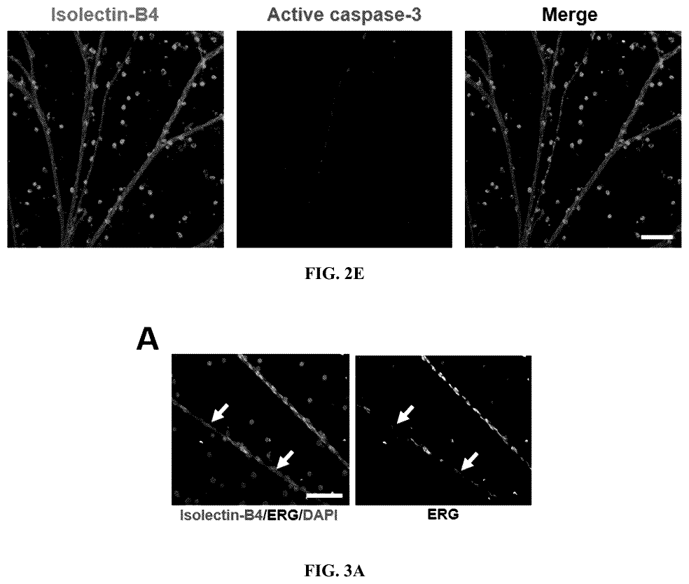

Coordinated constriction and apoptosis of hyaloid vessels excludes blood flow from actively regressing vessels. In order to investigate factors playing a role in physiological blood vessel regression, murine hyaloid vessels were dissected and imaged by flat mount as previously described³³, demonstrating a ˜50% decrease in vessel number between P4 and P8 (FIGS. 1A and B). Hyaloid vessel loss results from the apoptosis of ECs that line vascular luminal surfaces^{46,47} which can be visualized by terminal deoxynucleotidyl transferase dUTP nick end labeling (TUNEL) staining (FIG. 2A). From P4 to P8 the percentage of TUNEL⁺ hyaloid vessels increased from ˜5% to ˜25% (FIG. 2B), indicating that this is a useful experimental window in which to study vascular regression because it encompasses vessels at various stages of regression.

The TUNEL staining revealed a unique pattern in which dying cells were primarily clustered on distinct vascular branches (FIG. 2A). The same staining pattern was observed for active caspase-3, a more specific marker of the apoptotic cell death pathway (FIG. 2E). Meeson et al previously suggested that the segmental nature of hyaloid vessel apoptosis indicates that a coordinating factor like blood flow could synchronize EC death within a particular vascular branch³⁷. In support of this model, the inventors determined that at both postnatal day four (P4) and P8 the diameters of apoptotic vessel branches were significantly reduced compared to their non-apoptotic counterparts (FIG. 2C). In fact, from P4 to P8 there was a general reduction in the diameters of non-apoptotic vessels (from ˜15 μm to ˜10 μm), whereas apoptotic vessel diameters remained constant at ˜5-6 μm (FIG. 2C). Furthermore, immunostaining for the red blood cell (RBC) marker Ter119 demonstrated the exclusion of RBCs from constricted, apoptotic vessels (FIG. 2D). These data support published evidence of a correlation between the constriction and apoptosis of hyaloid vessels³⁷, altogether suggesting that the cessation of blood flow is an important factor regulating hyaloid vessel regression.

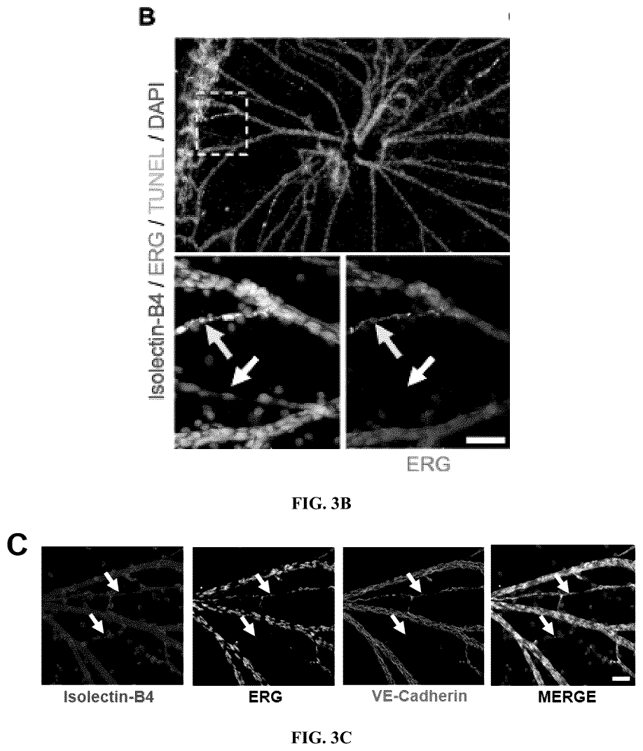

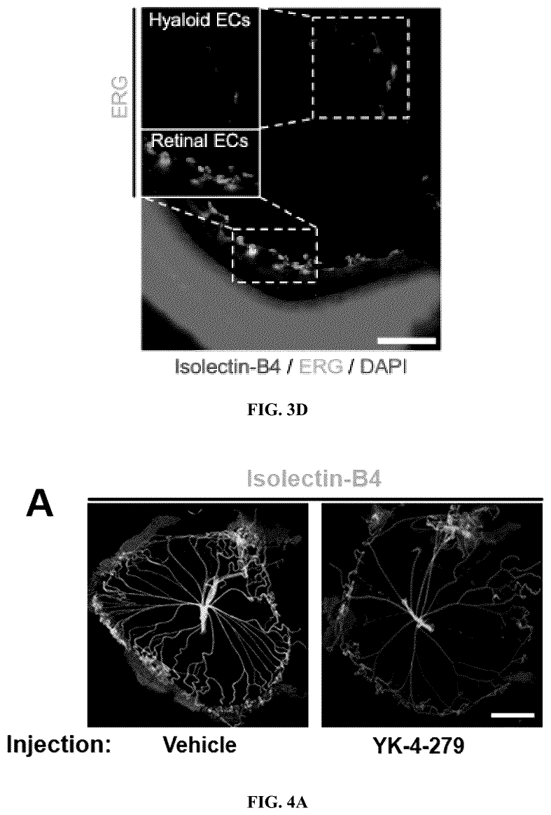

ETS-Related Gene (ERG) is downregulated in constricted regressing hyaloid vessels. ERG is an EC-specific ETS family transcription factor that has been reported to promote vascular regression when genetically deleted in mice⁴⁸. The inventors used immunofluorescence to compare the expression of ERG between hyaloid vessels at various stages of regression. Interestingly, ERG expression was notably absent from highly constricted hyaloid vessels when compared with adjacent non-constricted vascular branches (FIG. 3A). Moreover, ERG downregulation was observed in constricted vessels that were not yet apoptotic (as measured by TUNEL stain), suggesting ERG downregulation may play a transcriptional role in driving vessel regression (FIG. 3B). Indeed, it has been reported that inhibition of ERG results in EC apoptosis due to the transcriptional downregulation of the ERG target gene Cdh5, which encodes the adhesion molecular vascular endothelial (VE)-cadherin⁴⁹. Intriguingly, the inventors observed downregulation of VE-cadherin along with ERG in constricted hyaloid vessels (FIG. 3C), raising the possibility that a similar mechanism may function during hyaloid regression.

In both mice and humans, regression of the hyaloid vessels within the vitreous coincides with robust angiogenesis of ECs located on the superficial retinal surface that faces the vitreous⁵⁰. The spatiotemporal proximity of these vessel networks therefore stands in contrast to their opposing vascular fates. The inventors used immunofluorescence to compare expression of ERG within these two EC populations in cross sections of eyes from P8 wild-type mice. Whereas angiogenic retinal ECs were marked by robust ERG expression, regressive hyaloid ECs showed little ERG expression, suggesting ERG downregulation may play a role in distinguishing the behavior of these two vessel networks (FIG. 3D).

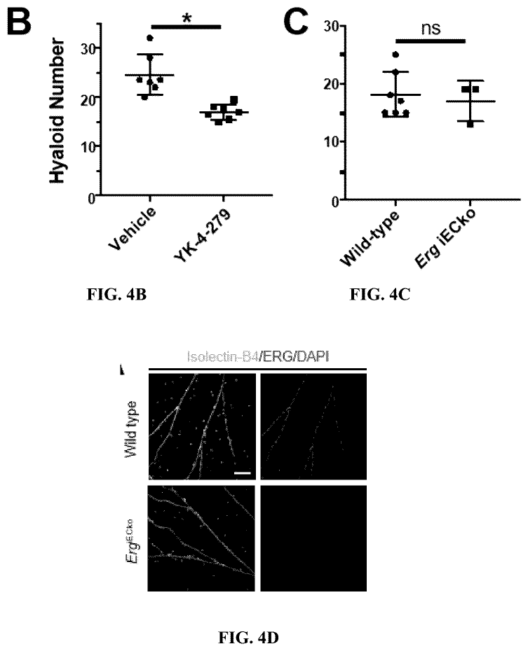

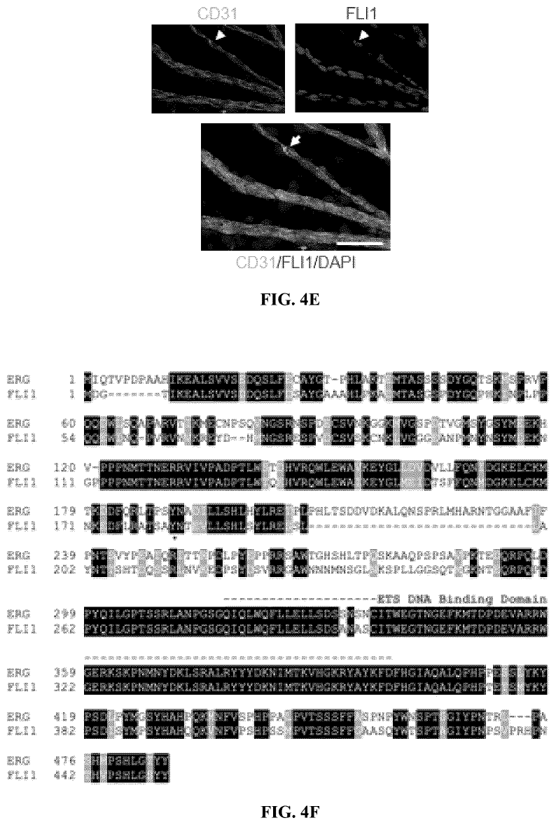

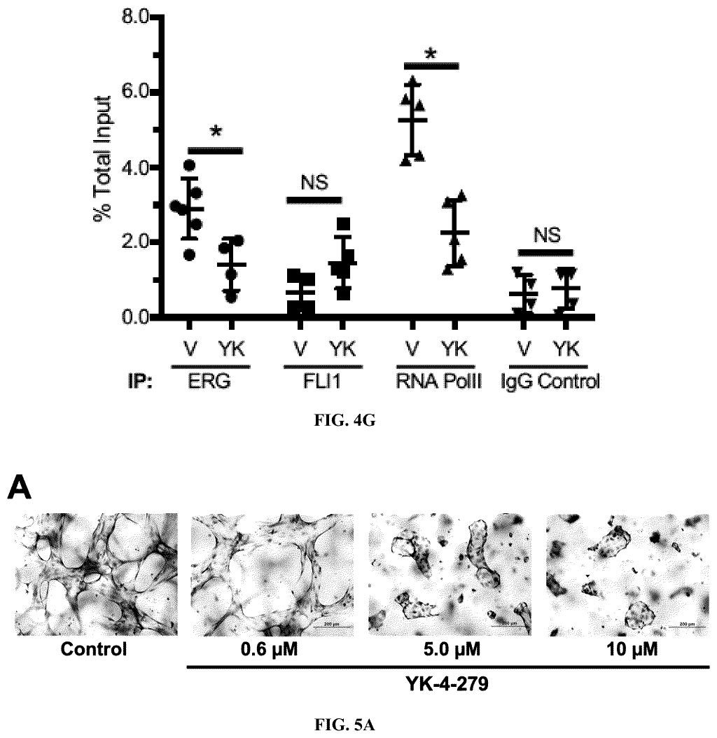

Pharmacological inhibition of endothelial ETS transcription factors promotes hyaloid regression. To better understand the role of ERG downregulation in hyaloid regression, intravitreally administered YK-4-279 was used, which is a small molecule inhibitor of ETS family transcription factors⁵¹, to P5 pups and assessed hyaloid vessel numbers and P7. Compared to vehicle-injected littermate controls, YK-4-279 injection resulted in a ˜40% reduction in hyaloid vessel number, which is consistent with a pro-regressive effect on the hyaloid vasculature (FIGS. 4A and B).

In addition to ERG, YK-4-279 likewise inhibits the structurally related transcription factor FLU (62% identical and 73% similar), which is also expressed in hyaloid ECs (FIG. 4E)⁵²⁻⁵⁴. To determine whether the pro-regressive effects of YK-4-279 could be assigned solely to the inhibition of ERG, the hyaloid vessel regression in mice was quantified following genetic deletion of Erg in ECs. The inventors crossed Erg^{flox} mice to the tamoxifen-inducible endothelial Cdh5(PAC)-Cre^{ERT2} line to generate Erg^{fl/fl};Cdh5(PAC)-Cre^{ERT2} (Erg^{iECko}) mice and administered tamoxifen at P4-P6 to delete Erg in ECs. Despite efficient Erg deletion (FIG. 4D), no differences were observed in hyaloid vessel number between Erg^{iECko} mice and their littermate controls at P7 (FIG. 4C). Therefore, the pro-regressive effects of YK-4-279 results from the broader inhibition of ETS family transcription factors in hyaloid vessels, rather than from the specific inhibition of ERG. This is consistent with previous reports demonstrating transcriptional compensation between ERG and FLI1⁵⁵⁻⁵⁷. FIG. 4G it shows that YK-4-279 blocks binding of ERG and of RNA Polymerase II to the promoter of the Cdh5 gene (VE-Cadherin) thereby demonstrating the YK-4-279 blocks ETS factor-mediated transcription in endothelial cells.

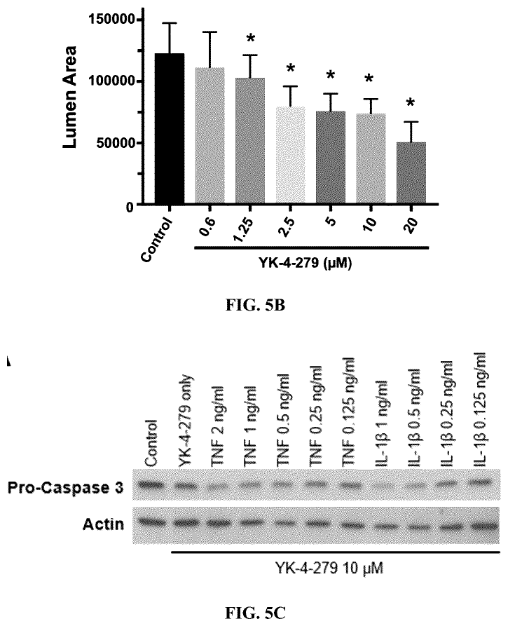

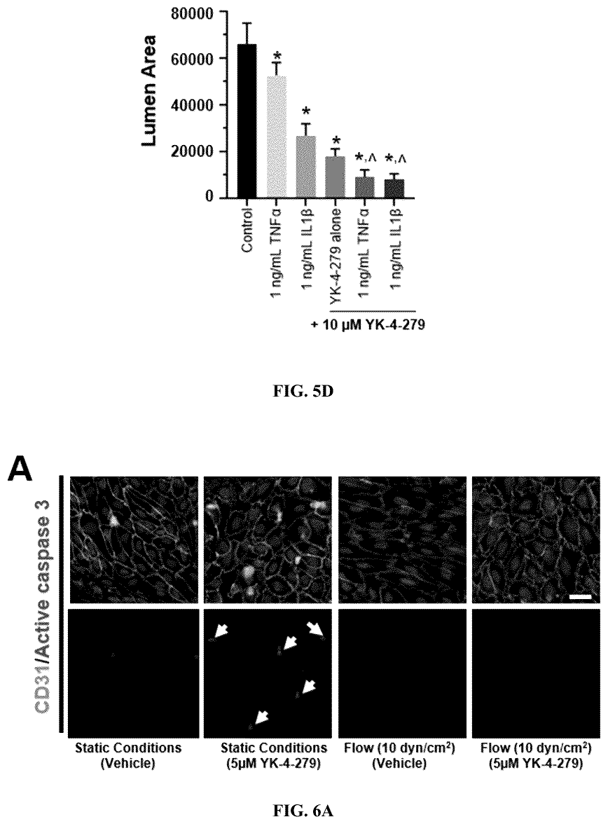

YK-4-279 promotes flow-dependent vascular regression in vitro. To further characterize its pro-regressive potential, YK-4-279 was added to pre-established three-dimensional (3D) cultures of Human Umbilical Vein Endothelial Cells (HUVECs). Importantly, this culture model recapitulates many aspects of in vivo vessel morphology and has previously been used to model vascular regression⁴⁰. Treatment with YK-4-279 resulted in a significant, dose-dependent loss of vascular luminal area at concentrations greater than 1.25 μM (FIGS. 5A and B), demonstrating pro-regressive properties of YK-4-279 in vitro that complement the effects observed on hyaloid vessels. FIG. 5C shows the potentiation of YK-4-279-induced regression by inflammatory cytokines. FIG. 5C is a Western blot of 3D HUVEC cultures treated with the indicated YK-4-279, TNFα, and IL1β concentrations for pro-caspase 3 and actin. Reduced pro-caspase 3 signal following YK-4-279 treatment indicates elevated EC apoptosis that is further increased by co-treatment with low concentrations of both TNFα and IL1β. (FIG. 5D) Quantification of 3D HUVEC lumen area under the indicated YK-4-279, TNFα, and IL1β concentrations. Co-incubation of TNFα or IL1β with YK-4-279 further increases the extent of vascular regression in vitro. *P<0.05 (versus Control, unpaired Student t-test), \hat{ }P<0.05 (versus YK-4-279 alone, unpaired Student t-test).

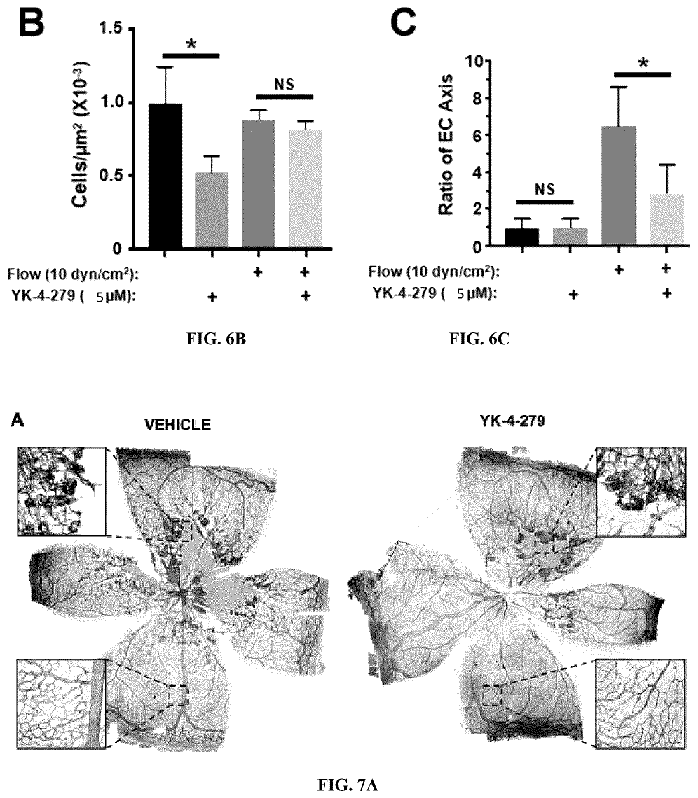

HUVECs grown in this 3D culture model form vascular lumens despite the absence of blood flow. The substantial pro-regressive effects of YK-4-279 in this model, taken together with the constriction of regressing hyaloid vessels, raises the possibility that the absence of blood flow is an important pre-requisite for YK-4-279-induced vascular regression. This possibility is supported by a recent report that the transcriptional consequences of ERG inhibition are greatly mitigated in ECs exposed to sheer stress⁵⁸. To test the effect of flow on YK-4-279-mediated pro-regressive effects, the inventors treated HUVECs with YK-4-279 in a flow chamber system. The inventors analyzed cells grown for 24 hrs under static conditions versus flow rates of 10 dyn/cm², which is comparable to the flow rate seen in perfused capillaries like the hyaloids⁵⁹. Under static conditions, treatment with 5 μM YK-4-279 resulted in significantly reduced cell numbers and increased active caspase-3 staining (FIGS. 6A and B), indicating an increase in EC apoptosis. In contrast, under flow conditions YK-4-279 treatment had no apparent effect on cell number or on active caspase-3 staining (FIGS. 6A and B), demonstrating the protective effect of flow on YK-4-279-mediated EC death in vitro. Interestingly, YK-4-279 treatment inhibited the alignment of HUVECs in the direction of flow. YK-4-279-treated HUVECs had a more cobblestone shape under flow conditions when compared to vehicle-treated cells. Therefore, YK-4-279 still mediated effects on EC behavior under flow conditions, although it failed to drive EC death under flow.

YK-4-279 induces regression of retinal neovessels following oxygen-induced retinopathy. Oxygen-induced retinopathy (OIR) has been established as a useful in vivo model of ROP that recapitulates the NV component of both ROP and DR^{44,45}. Briefly, P7 wild type mice are transferred to hyperoxic conditions (75% O₂) for 5 d, which results in vaso-obliteration of the central retina. At P12 mice are returned to room air (21% O₂), which results in retinal hypoxia and retinal neovascularization that peaks at P17-18. Importantly, due to their tortuous and disorganized structure, retinal neovessels are poorly perfused relative to healthy retinal vessels, suggesting that they may be uniquely susceptible to YK-4-279-induced regression⁶⁰.

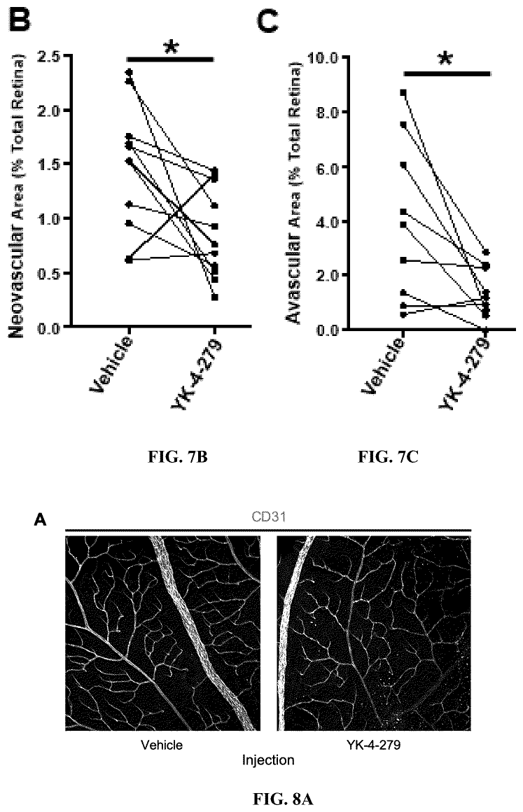

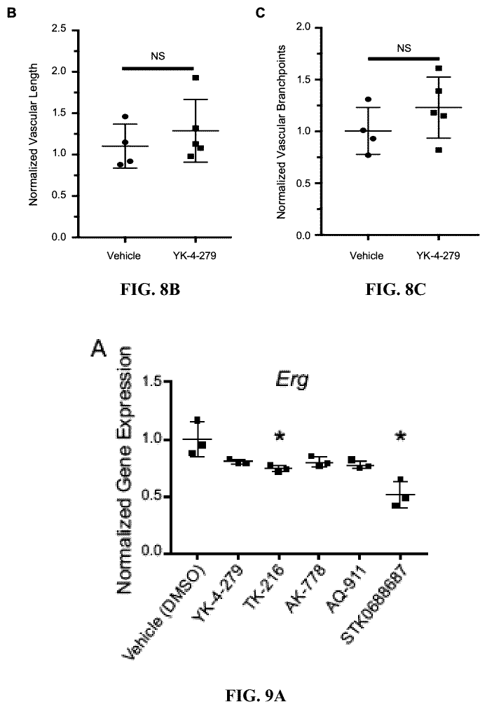

To test this possibility, P18 mice that had been subjected to the OIR protocol were given intravitreal injections of YK-4-279 in one eye and a vehicle control in their contralateral eye. Two days after the injection, P20 mice were euthanized and retinas were dissected and immunostained for CD31 to quantify retinal vascular area as previously described⁴⁵. YK-4-279 injection resulted in a decrease in retinal neovascular area in 9 out of 11 mice, with only one mouse showing an increase in neovessels in the inhibitor-injected eye relative to the control eye. Altogether, a significant ˜40% reduction in retinal neovascular area with inhibitor treatment (FIGS. 7A and B) was observed. Importantly, YK-4-279 did not appear to adversely affect healthy retinal vessels located at the periphery of the retina. Moreover, intraocular injection of adult wild-type mice with YK-4-279 showed no effects on retinal vascularization (Montoya-Zegarra, J. A., et al., AutoTube: a novel software for the automated morphometric analysis of vascular networks in tissues. Angiogenesis. 22, 223-236 (2019)) (FIG. 8A-8C). The inventors conclude that the pro-regressive effects of YK-4-279 are unique to the poorly perfused retinal vessels generated in the OIR model but are not seen in normal retinal vessels. Finally, the inventors also observed a ˜60% decrease in the retinal avascular area with YK-4-279 treatment in the OIR model. Therefore, treatment with the inhibitor resulted in a surprising overall increase in retinal vascular compared to treatment with a vehicle control in the OIR model.

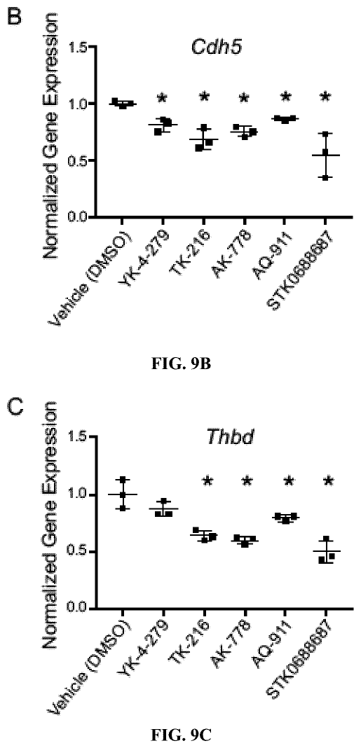

FIGS. 9A to 9C are graphs that show the transcriptional downregulation of the ERG target genes Erg (autoregulation; FIG. 9A), Cdh5 (FIG. 9B), and Thbd (FIG. 9C) in Human Umbilical Vein Endothelial Cells (HUVECs) treated with the indicated inhibitors (5 μM) for 8 h. Gene expression was normalized to three housekeeping genes (Actin, Gapdh, and Rn18s). *p<0.05 compared to Vehicle (Student's two-tailed t-test).

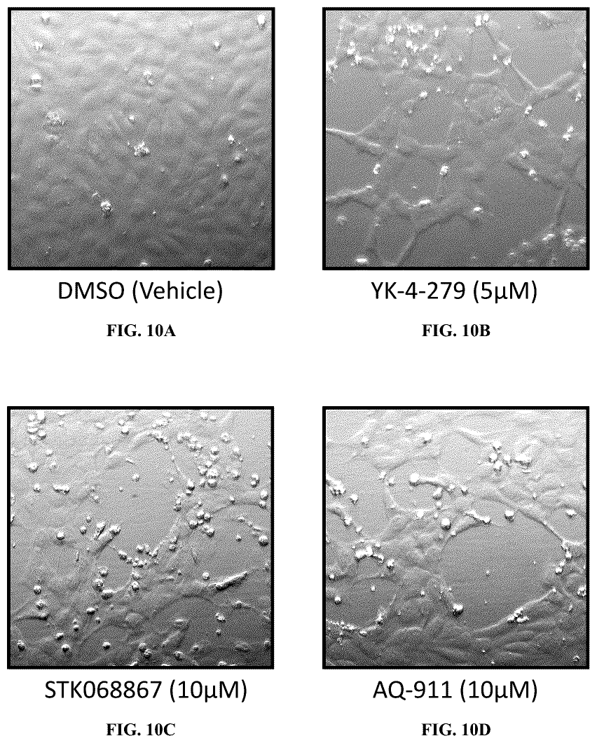

FIGS. 10A to 10D show that human umbilical vein endothelial cells were treated with the indicated inhibitor concentrations for 24 hr prior to phase contrast imaging. Both STK068867 and AQ-911 treatment result in loss of cell density suggesting the promotion of cell death as observed for YK-4-279.

Cardiovascular function requires complex, organ-specific vascular patterning achieved through the continuous integration of pro- and anti-growth signals. This has led to the recognition of well-established angiogenic pathways that coordinate vascular development and maintenance. However, comparatively little is known about molecular pathways that promote the regression of pre-existing vessels²⁷. This is partly due to the paucity of naturally occurring examples of vascular regression. Indeed, much of the literature devoted to the subject refers to the pruning of dispensable vessels during vessel network maturation³⁰. However, this is often a non-apoptotic^{61,62} process limited to a small percentage of cells within a vessel network and is therefore not associated with substantial changes in tissue vascularization.

In contrast, a small number of physiological processes have been documented in which the complete involution of a pre-established, functional vascular network is brought about over a short period of time. Among these are luteolysis in the adult ovarian cycle⁶³ and the developmental regression of a small number of ocular blood supplies, including the hyaloid vessels²⁸. Although rare, regression of this form is uniquely poised to offer new insights into the therapeutic induction of vascular regression in cases of pathological hypervascularization. The eye appears to be particularly susceptible to vascular misregulation, as evidenced by a number of ocular pathologies with well-documented vascular abnormalities, including ROP and DR^{2,4}. This susceptibility may reflect the delicate balance required for satisfying the high nutritional demands of the retina against the physical impediment that blood vessels impose on light transmission. The hyaloids are therefore a unique and experimentally tractable system in which to identify pro-regressive molecular pathways relevant to the treatment of ocular NV diseases.

To date, studies about factors that contribute to hyaloid regression have identified pro-regressive cues linked to Wnt ligands³³, Angiopoietin-2⁶⁴, pro-inflammatory cytokines and thrombin⁴⁰, VEGF depletion⁴⁷, and blood flow cessation³⁷. However, many of these stimuli also play important functions in non-regressive contexts, suggesting that few external stimuli are sufficient for the initiation of regression by themselves. It was found herein that the ECs of regressive hyaloid vessels are marked by the loss of nuclear ERG expression provides a novel endothelial phenotype associated with vascular regression. ERG expression is primarily restricted to ECs, where it regulates a large number of endothelial genes^{48,65}. Embryonic deletion of Erg is lethal, although its postnatal deletion is associated with more subtle phenotypes⁶⁶. One likely explanation for this temporal difference in phenotypic severity is the acquisition of functional redundancy with other endothelial ETS family transcription factors, such as FLI1, with which ERG shares structure, endothelial specificity, and many target genes^{55,56}.

ETS factor binding to the DNA motif GGAA/T results in the transcriptional regulation of many genes, which complicates the determination of specific gene target(s) that mediate the pro-regressive effects of ERG/FLI1 inhibition with YK-4-279. Furthermore, ERG/FLI1 transcriptionally regulate many pro-survival pathways that play roles in vessel stability^{67,68}. Therefore, it seems likely that complex transcriptional effects account for the pro-regressive effects of YK-4-279.

ERG is robustly expressed in most endothelial populations (for which reason it is commonly employed as an EC nuclear marker) and has only been observed to be downregulated under specific conditions^{55,69}. These studies reported transcriptional repression of ERG in vitro by inflammatory cytokines⁶⁹. The inventors have recently demonstrated a pro-regressive role of inflammatory cytokines in hyaloid regression⁴⁰. In addition, the coordination of ERG downregulation along constricted hyaloid vessels shows flow-dependent regulation of ERG expression. By way of explanation, but in no way a limitation of the present invention, the inventors hypothesize that the loss of blood flow and downregulation of ERG are independent events, thereby providing a multi-factor check against unwanted regressive events which are costly and irreversible. Moreover, ensuring a lack of blood flow prior to the onset of regression likely prevents intraocular hemorrhage at sites of EC apoptosis.

By way of explanation, and in no way a limitation of the present invention, the mechanism by which YK-4-279 specifically promotes regression of slow-flow vessels and spares vessels with normal blood flow, the inventors hypothesize that interactions between ETS factors and Krüppel-like factor (KLF) proteins are important. KLF2 and KLF4 are transcription factors that are upregulated in endothelial cells subjected to flow and that promote the expression of cell survival genes⁷⁰. However, under slow-flow conditions, the ETS factor ERG must first bind the promoter of the pro-survival gene thrombomodulin (Thbd) and then directly recruit KLF2 to that promoter to drive transcription⁷¹. The same study showed ERG also promotes pro-transcriptional histone acetylation and chromatin opening at the Thbd promoter under slow-flow conditions. However, under normal flow conditions, when KLF proteins are expressed at higher levels, ERG is not needed for KLF2 recruitment or for chromatin-opening at the Thbd promoter and does not impact Thbd expression. Again, by way of explanation, the inventors postulate that ETS factors are required for recruiting KLF proteins, opening chromatin, and promoting transcription of pro-survival genes under slow-flow conditions, but the upregulation of KLF proteins supplants the need for ETS factors in these roles under normal flow conditions. The ETS inhibitors described herein, such as YK-4-279, disrupt the pioneering activities of ETS factors (i.e., DNA binding and subsequent recruitment of KLFs and chromatin-remodeling factors) at pro-survival genes under slow-flow but would be inconsequential under normal flow conditions in which KLF proteins do not rely on ETS factors for opening chromatin and promoting pro-survival genes. Finally, the inventors further postulate that inhibitors of ETS:KLF interactions would likewise drive vascular regression by disrupting the transcription of pro-survival genes under slow-flow conditions.

The specificity of YK-4-279-induced regression to low sheer stress conditions affords a unique opportunity for the treatment of ocular NV disorders. Due to their tortuous and disorganized structure, retinal neovessels are poorly perfused. The inventors recognized that these vessels, and not their healthy counterparts, would be uniquely susceptible to ETS inhibitors, e.g., YK-4-279-induced regression. Indeed, YK-4-279 injection resulted in a significant improvement in retinal vascular structure in the OIR model. For example, a significant reduction in retinal NV tufts driven by an apparent increase in EC apoptosis was observed. Importantly, YK-4-279 failed to affect healthy vessels in the OIR model or in normal adult eyes, further suggesting that flow confers protection to the regressive effects of the inhibitor as the inventors had seen in vitro. The observation that YK-4-279 treatment additionally resulted in a decrease in retinal avascular area was surprising and unexpected. If left untreated, OIR-induced NVs spontaneously regress around P25, and the avascular central retina eventually becomes revascularized⁴⁵. Therefore, YK-4-279 treatment may simply speed up these processes in the OIR-induced NVs. YK-4-279 may also stimulate vascular growth.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±0.1, 0.5, 1, 2, 3, 4, 5, 6, 7, 10, 12 or 15%, or as understood to be within a normal tolerance in the art, for example, within 2 standard deviations of the mean. Unless otherwise clear from the context, all numerical values provided herein are modified by the term about.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1 Wong-Riley, M. Energy metabolism of the visual system. Eye Brain. 2:99-116 (2010).
2 Campochiaro, P. A. Ocular neovascularization. J Mol Med (Berl) 91, 311-321, doi:10.1007/s00109-013-0993-5 (2013).
3 Shah, P. K. et al. Retinopathy of prematurity: Past, present and future. World J Clin Pediatr 5, 35-46, doi:10.5409/wjcp.v5.i1.35 (2016).
4 Lee, P., Wang, C. C. & Adamis, A. P. Ocular neovascularization: an epidemiologic review. Surv Ophthalmol 43, 245-269, doi:10.1016/s0039-6257(98)00035-6 (1998).
5 Kim, M. J., Kim, S. J. & Yu, Y. S. The risk for retinal detachment associated with hemorrhages pre- and postlaser treatment in retinopathy of prematurity. Retina 28, 1451-1457, doi:10.1097/iae.0b013e31817f2f15 (2008).
6 Hutcheson, K. A., Nguyen, A. T., Preslan, M. W., Ellish, N. J. & Steidl, S. M. Vitreous hemorrhage in patients with high-risk retinopathy of prematurity. Am J Ophthalmol 136, 258-263, doi:10.1016/s0002-9394(03)00190-9 (2003).
7 Lad, E. M., Hernandez-Boussard, T., Morton, J. M. & Moshfeghi, D. M. Incidence of retinopathy of prematurity in the United States: 1997 through 2005. Am J Ophthalmol 148, 451-458, doi:10.1016/j.ajo.2009.04.018 (2009).
8 Fong, D. S. et al. Retinopathy in diabetes. Diabetes Care 27 Suppl 1, S84-87, doi:10.2337/diacare.27.2007.s84 (2004).
9 Photocoagulation treatment of proliferative diabetic retinopathy. Clinical application of Diabetic Retinopathy Study (DRS) findings, DRS Report Number 8. The Diabetic Retinopathy Study Research Group. Ophthalmology 88, 583-600 (1981).
10 Early Treatment For Retinopathy Of Prematurity Cooperative, G. Revised indications for the treatment of retinopathy of prematurity: results of the early treatment for retinopathy of prematurity randomized trial. Arch Ophthalmol 121, 1684-1694, doi:10.1001/archopht.121.12.1684 (2003).
11 Repka, M. X. et al. Outcome of eyes developing retinal detachment during the Early Treatment for Retinopathy of Prematurity Study (ETROP). Arch Ophthalmol 124, 24-30, doi:10.1001/archopht.124.1.24 (2006).
12 Mohamed, Q., Gillies, M. C. & Wong, T. Y. Management of diabetic retinopathy: a systematic review. JAMA 298, 902-916, doi:10.1001/jama.298.8.902 (2007).
13 Cheung, N., Mitchell, P. & Wong, T. Y. Diabetic retinopathy. Lancet 376, 124-136, doi:10.1016/50140-6736(09)62124-3 (2010).
14 Wu, W. C. et al. An updated study of the use of bevacizumab in the treatment of patients with prethreshold retinopathy of prematurity in taiwan. Am J Ophthalmol 155, 150-158 e151, doi:10.1016/j.ajo.2012.06.010 (2013).
15 Chen, S. N. et al. Intravitreal anti-vascular endothelial growth factor treatment for retinopathy of prematurity: comparison between Ranibizumab and Bevacizumab. Retina 35, 667-674, doi:10.1097/IAE.0000000000000380 (2015).
16 Mintz-Hittner, H. A., Kennedy, K. A., Chuang, A. Z. & Group, B.-R. C. Efficacy of intravitreal bevacizumab for stage 3+ retinopathy of prematurity. N Engl J Med 364, 603-615, doi:10.1056/NEJMoa1007374 (2011).
17 Kimoto, K. & Kubota, T. Anti-VEGF Agents for Ocular Angiogenesis and Vascular Permeability. J Ophthalmol 2012, 852183, doi:10.1155/2012/852183 (2012).
18 Ozaki, H. et al. Blockade of vascular endothelial cell growth factor receptor signaling is sufficient to completely prevent retinal neovascularization. Am J Pathol 156, 697-707, doi:10.1016/S0002-9440(10)64773-6 (2000).
19 Pieh, C. et al. VEGF-A, VEGFR-1, VEGFR-2 and Tie2 levels in plasma of premature infants: relationship to retinopathy of prematurity. Br J Ophthalmol 92, 689-693, doi:10.1136/bjo.2007.128371 (2008).
20 Wu, W. C. et al. Serum Vascular Endothelial Growth Factor after Bevacizumab or Ranibizumab Treatment for Retinopathy of Prematurity. Retina 37, 694-701, doi:10.1097/IAE.0000000000001209 (2017).
21 Hu, J. et al. Reactivation of retinopathy of prematurity after bevacizumab injection. Arch Ophthalmol 130, 1000-1006, doi:10.1001/archophthalmol.2012.592 (2012).
22 Wong, R. K., Hubschman, S. & Tsui, I. Reactivation of retinopathy of prematurity after ranibizumab treatment. Retina 35, 675-680, doi:10.1097/IAE.0000000000000578 (2015).
23 Lepore, D. et al. Intravitreal bevacizumab versus laser treatment in type 1 retinopathy of prematurity: report on fluorescein angiographic findings. Ophthalmology 121, 2212-2219, doi:10.1016/j.ophtha.2014.05.015 (2014).
24 Lepore, D. et al. Follow-up to Age 4 Years of Treatment of Type 1 Retinopathy of Prematurity Intravitreal Bevacizumab Injection versus Laser: Fluorescein Angiographic Findings. Ophthalmology 125, 218-226, doi:10.1016/j.ophtha.2017.08.005 (2018).
25 Chandra, S., Sheth, J., Anantharaman, G. & Gopalakrishnan, M. Ranibizumab-induced retinal reperfusion and regression of neovascularization in diabetic retinopathy: An angiographic illustration. Am J Ophthalmol Case Rep 9, 41-44, doi:10.1016/j.ajoc.2018.01.006 (2018).
26 Writing Committee for the Diabetic Retinopathy Clinical Research, N. et al. Panretinal Photocoagulation vs Intravitreous Ranibizumab for Proliferative Diabetic Retinopathy: A Randomized Clinical Trial. JAMA 314, 2137-2146, doi:10.1001/jama.2015.15217 (2015).
27 Korn, C. & Augustin, H. G. Mechanisms of Vessel Pruning and Regression. Dev Cell 34, 5-17, doi:10.1016/j.devcel.2015.06.004 (2015).
28 Ito, M. & Yoshioka, M. Regression of the hyaloid vessels and pupillary membrane of the mouse. Anat Embryol (Berl) 200, 403-411, doi:10.1007/s004290050289 (1999).
29 Modlich, U., Kaup, F. J. & Augustin, H. G. Cyclic angiogenesis and blood vessel regression in the ovary: blood vessel regression during luteolysis involves endothelial cell detachment and vessel occlusion. Lab Invest 74, 771-780 (1996).
30 Ricard, N. & Simons, M. When it is better to regress: dynamics of vascular pruning. PLoS Biol 13, e1002148, doi:10.1371/journal.pbio.1002148 (2015).
31 Silbert, M. & Gurwood, A. S. Persistent hyperplastic primary vitreous. Clin Eye Vis Care 12, 131-137, doi:10.1016/s0953-4431(00)00054-0 (2000).
32 Lang, R. A. & Bishop, J. M. Macrophages are required for cell death and tissue remodeling in the developing mouse eye. Cell 74, 453-462, doi:10.1016/0092-8674(93)80047-i (1993).
33 Lobov, I. B. et al. WNT7b mediates macrophage-induced programmed cell death in patterning of the vasculature. Nature 437, 417-421, doi:10.1038/nature03928 (2005).
34 Nayak, G. et al. Developmental vascular regression is regulated by a Wnt/beta-catenin, MYC and CDKN1A pathway that controls cell proliferation and cell death. Development 145, doi:10.1242/dev.154898 (2018).
35 Parr, B. A., Shea, M. J., Vassileva, G. & McMahon, A. P. Mouse Wnt genes exhibit discrete domains of expression in the early embryonic CNS and limb buds. Development 119, 247-261 (1993).
36 Stenman, J. M. et al. Canonical Wnt signaling regulates organ-specific assembly and differentiation of CNS vasculature. Science 322, 1247-1250, doi:10.1126/science.1164594 (2008).
37 Meeson, A., Palmer, M., Calfon, M. & Lang, R. A relationship between apoptosis and flow during programmed capillary regression is revealed by vital analysis. Development 122, 3929-3938 (1996).
38 Yoshikawa, Y. et al. Developmental regression of hyaloid vasculature is triggered by neurons. J Exp Med 213, 1175-1183, doi:10.1084/jem.20151966 (2016).
39 Hackett, S. F., Wiegand, S., Yancopoulos, G. & Campochiaro, P. A. Angiopoietin-2 plays an important role in retinal angiogenesis. J Cell Physiol 192, 182-187, doi:10.1002/jcp.10128 (2002).
40 Koller, G. M. et al. Proinflammatory Mediators, IL (Interleukin)-1beta, TNF (Tumor Necrosis Factor) alpha, and Thrombin Directly Induce Capillary Tube Regression. Arterioscler Thromb Vasc Biol 40, 365-377, doi:10.1161/ATVBAHA.119.313536 (2020).
41 Wang, Y. et al. Ephrin-B2 controls VEGF-induced angiogenesis and lymphangiogenesis. Nature 465, 483-486, doi:10.1038/nature09002 (2010).
42 Koh, W., Stratman, A. N., Sacharidou, A. & Davis, G. E. In vitro three dimensional collagen matrix models of endothelial lumen formation during vasculogenesis and angiogenesis. Methods Enzymol 443, 83-101, doi:10.1016/S0076-6879(08)02005-3 (2008).
43 Stratman, A. N., Davis, M. J. & Davis, G. E. VEGF and FGF prime vascular tube morphogenesis and sprouting directed by hematopoietic stem cell cytokines. Blood 117, 3709-3719, doi:10.1182/blood-2010-11-316752 (2011).
44 Smith, L. E. et al. Oxygen-induced retinopathy in the mouse. Invest Ophthalmol Vis Sci 35, 101-111 (1994).
45 Connor, K. M. et al. Quantification of oxygen-induced retinopathy in the mouse: a model of vessel loss, vessel regrowth and pathological angiogenesis. Nat Protoc 4, 1565-1573, doi:10.1038/nprot.2009.187 (2009).
46 Zhu, M. et al. The human hyaloid system: cell death and vascular regression. Exp Eye Res 70, 767-776, doi:10.1006/exer.2000.0844 (2000).
47 Meeson, A. P., Argilla, M., Ko, K., Witte, L. & Lang, R. A. VEGF deprivation-induced apoptosis is a component of programmed capillary regression. Development 126, 1407-1415 (1999).
48 Shah, A. V., Birdsey, G. M. & Randi, A. M. Regulation of endothelial homeostasis, vascular development and angiogenesis by the transcription factor ERG. Vascul Pharmacol 86, 3-13, doi:10.1016/j.vph.2016.05.003 (2016).
49 Birdsey, G. M. et al. Transcription factor Erg regulates angiogenesis and endothelial apoptosis through VE-cadherin. Blood 111, 3498-3506, doi:10.1182/blood-2007-08-105346 (2008).
50 Gariano, R. F. & Gardner, T. W. Retinal angiogenesis in development and disease. Nature 438, 960-966, doi:10.1038/nature04482 (2005).
51 Tomlins, S. A. et al. Recurrent fusion of TMPRSS2 and ETS transcription factor genes in prostate cancer. Science 310, 644-648, doi:10.1126/science.1117679 (2005).
52 Rahim, S. et al. YK-4-279 inhibits ERG and ETV1 mediated prostate cancer cell invasion. PLoS One 6, e19343, doi:10.1371/journal.pone.0019343 (2011).
53 Winters, B. et al. Inhibition of ERG Activity in Patient-derived Prostate Cancer Xenografts by YK-4-279. Anticancer Res 37, 3385-3396, doi:10.21873/anticanres.11705 (2017).
54 Barber-Rotenberg, J. S. et al. Single enantiomer of YK-4-279 demonstrates specificity in targeting the oncogene EWS-FLI1. Oncotarget 3, 172-182, doi:10.18632/oncotarget.454 (2012).
55 Looney, A. P. et al. Synergistic Role of Endothelial ERG and FLI1 in Mediating Pulmonary Vascular Homeostasis. Am J Respir Cell Mol Biol 57, 121-131, doi:10.1165/rcmb.2016-0200OC (2017).
56 Nagai, N. et al. Downregulation of ERG and FLU expression in endothelial cells triggers endothelial-to-mesenchymal transition. PLoS Genet 14, e1007826, doi:10.1371/journal.pgen.1007826 (2018).
57 Pham, V. N. et al. Combinatorial function of ETS transcription factors in the developing vasculature. Dev Biol 303, 772-783, doi:10.1016/j.ydbio.2006.10.030 (2007).
58 Peghaire, C. et al. The transcription factor ERG regulates a low shear stress-induced anti-thrombotic pathway in the microvasculature. Nat Commun 10, 5014, doi:10.1038/s41467-019-12897-w (2019).
59 Lipowsky, H. H. Microvascular rheology and hemodynamics. Microcirculation 12, 5-15, doi:10.1080/10739680590894966 (2005).
60 Hoang, M. V., Smith, L. E. & Senger, D. R. Calpain inhibitors reduce retinal hypoxia in ischemic retinopathy by improving neovascular architecture and functional perfusion. Biochim Biophys Acta 1812, 549-557, doi:10.1016/j.bbadis.2010.08.008 (2011).
61 Franco, C. A. et al. Dynamic endothelial cell rearrangements drive developmental vessel regression. PLoS Biol 13, e1002125, doi:10.1371/journal.pbio.1002125 (2015).
62 Kochhan, E. et al. Blood flow changes coincide with cellular rearrangements during blood vessel pruning in zebrafish embryos. PLoS One 8, e75060, doi: 10.1371/journal.pone.0075060 (2013).
63 Plendl, J. Angiogenesis and vascular regression in the ovary. Anat Histol Embryol 29, 257-266, doi:10.1046/j.1439-0264.2000.00265.x (2000).
64 Rao, S. et al. Obligatory participation of macrophages in an angiopoietin 2-mediated cell death switch. Development 134, 4449-4458, doi:10.1242/dev.012187 (2007).
65 Kalna, V. et al. The Transcription Factor ERG Regulates Super-Enhancers Associated With an Endothelial-Specific Gene Expression Program. Circ Res 124, 1337-1349, doi:10.1161/CIRCRESAHA.118.313788 (2019).
66 Birdsey, G. M. et al. The endothelial transcription factor ERG promotes vascular stability and growth through Wnt/beta-catenin signaling. Dev Cell 32, 82-96, doi:10.1016/j.devcel.2014.11.016 (2015).
67 Yuan, L. et al. ETS-related gene (ERG) controls endothelial cell permeability via transcriptional regulation of the claudin 5 (CLDNS) gene. J Biol Chem 287, 6582-6591, doi:10.1074/jbc.M111.300236 (2012).
68 Yuan, L. et al. RhoJ is an endothelial cell-restricted Rho GTPase that mediates vascular morphogenesis and is regulated by the transcription factor ERG. Blood 118, 1145-1153, doi:10.1182/blood-2010-10-315275 (2011).
69 Yuan, L. et al. Antiinflammatory effects of the ETS factor ERG in endothelial cells are mediated through transcriptional repression of the interleukin-8 gene. Circ Res 104, 1049-1057, doi:10.1161/CIRCRESAHA.108.190751 (2009).
70 Fan Y, Lu H, Liang W, Hu W, Zhang J and Chen Y E. Kruppel-like factors and vascular wall homeostasis. J Mol Cell Biol. 2017; 9:352-363.
71 Peghaire C, Dufton N P, Lang M, Salles C, II, Ahnstrom J, Kalna V, Raimondi C, Pericleous C, Inuabasi L, Kiseleva R, Muzykantov V R, Mason J C, Birdsey G M and Randi A M. The transcription factor ERG regulates a low shear stress-induced anti-thrombotic pathway in the microvasculature. Nat Commun. 2019; 10:5014.

1. A method of inducing vascular regression in poorly perfused blood vessels in a subject comprising providing the subject with an effective amount of an inhibitor of an Endothelial ETS Family Transcription Factor.

2. The method of claim 1, wherein the subject is in need of treatment for retinopathy of prematurity (ROP), diabetic retinopathy (DR), or vascular malformations.

3. The method of claim 1, wherein the inhibitor of an Endothelial ETS Family Transcription Factor is selected from at least one of:
an siRNA, RNAi, an RNAse inhibitor, or a small molecule inhibitor;
an RNA Helicase A inhibitor; or
is YK 4-279, TK216, or derivatives having the formula, respectively: 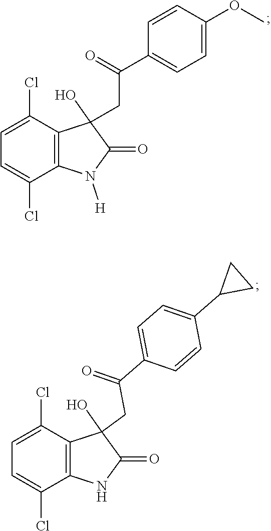 [SMILES: COc1ccc(C(=O)CC2(O)C(=O)Nc3c(Cl)ccc(Cl)c32)cc1 ; O=C(CC1(O)C(=O)Nc2c(Cl)ccc(Cl)c21)c1ccc(C2CC2)cc1]
or
the inhibitor has the formula: 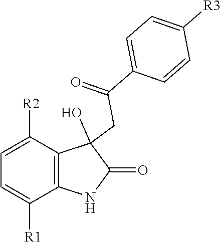
wherein, R1, R2, R3 are the same or different and are each independently hydrogen, halogen, Cl, Br, F, I, OH, a saturated or unsaturated alkyl group, a cycloalkyl group, a substituted or unsubstituted aryl group, an aryl group, an alkoxy group, a nitrone group or a selected from R′COO, R′COOCH(R″), R′CH═CH, R′CONN, R′CONH(R″); wherein R′, R″ are each independently a saturated or unsaturated alkyl, ring, an alkyl group, a substituted or unsubstituted aryl group, or an aromatic hetero group.

4. (canceled)

5. (canceled)

6. (canceled)

7. The method of claim 1, further comprising measuring vascular regression in poorly perfused blood vessels by hyaloid regression.

8. The method of claim 1, wherein the inhibitor of an Endothelial ETS Family Transcription Factor is administered topically, subconjunctivally, intracamerally, subtenonally, subretinally, subchoroidally, suprachoroidally, supraorbitally, retrobulbarlly, as an ocular implant, or intravitreally, and wherein the composition is an eye drop, gel, ointment, spray, a reservoir, or mist.

9. The method of claim 1, wherein the inhibitor of an Endothelial ETS Family Transcription Factor at least one of: decrease retinal neovessels or vascular malformations by at least 40% or a retinal avascular area by at least 60% compared to a vehicle-injected contralateral eye.

10. The method of claim 1, wherein the inhibitor of an Endothelial ETS Family Transcription Factor does not inhibit vascular endothelial growth factor (VEGF).

11. A method of inducing vascular regression in poorly perfused blood vessels comprising:
identifying a subject in need of treatment for neovascularization; and
providing the subject with an effective amount of an inhibitor of an Endothelial ETS Family Transcription Factor.

12. The method of claim 11, wherein the subject is in need of treatment for retinopathy of prematurity (ROP), diabetic retinopathy (DR), or vascular malformations.

13. The method of claim 11, wherein the inhibitor of an Endothelial ETS Family Transcription Factor is selected from at least one of:
an siRNA, RNAi, an RNAse inhibitor, or a small molecule inhibitor;
an RNA Helicase A inhibitor; or
is YK 4-279, TK216, or derivatives having the formula, respectively: 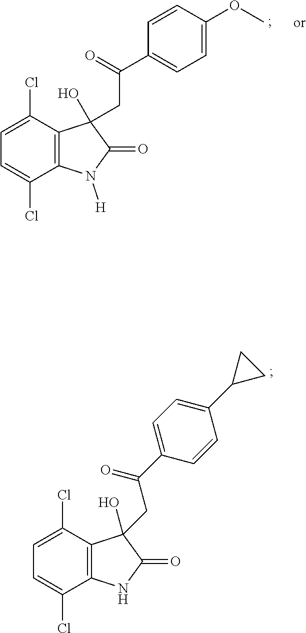 [SMILES: COc1ccc(C(=O)CC2(O)C(=O)Nc3c(Cl)ccc(Cl)c32)cc1 ; O=C(CC1(O)C(=O)Nc2c(Cl)ccc(Cl)c21)c1ccc(C2CC2)cc1]
or
has the formula: 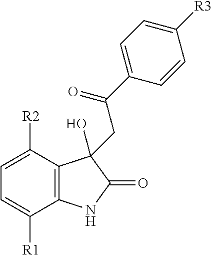
wherein, R1, R2, R3 are the same or different and are each independently hydrogen, halogen, Cl, Br, F, I, OH, a saturated or unsaturated alkyl group, a cycloalkyl group, a substituted or unsubstituted aryl group, an aryl group, an alkoxy group, a nitrone group or a selected from R′COO, R′COOCH(R″), R′CH═CH, R′CONH, R′CONH(R″); wherein R′, R″ are each independently a saturated or unsaturated alkyl, ring, an alkyl group, a substituted or unsubstituted aryl group, or an aromatic hetero group.

14. (canceled)

15. (canceled)

16. (canceled)

17. The method of claim 11, further comprising measuring vascular regression in poorly perfused blood vessels by hyaloid regression.

18. The method of claim 11, wherein the inhibitor of an Endothelial ETS Family Transcription Factor is administered topically, subconjunctivally, intracamerally, subtenonally, subretinally, subchoroidally, suprachoroidally, supraorbitally, retrobulbarlly, as an ocular implant, or intravitreally, and wherein the composition is an eye drop, gel, ointment, spray, a reservoir, or mist.

19. The method of claim 11, wherein the inhibitor of an Endothelial ETS Family Transcription Factor at least one of: decrease retinal neovessels or vascular malformations by at least 40% or a retinal avascular area by at least 60% compared to a vehicle-injected contralateral eye.

20. The method of claim 11, wherein the inhibitor of an Endothelial ETS Family Transcription Factor does not inhibit vascular endothelial growth factor (VEGF).

21. A method for treating a retinopathy of prematurity (ROP), diabetic retinopathy (DR), or vascular malformation patient with inhibitor of an Endothelial ETS Family Transcription Factor, the method comprising the steps of:
performing or having performed a vascular regression analysis in a poorly perfused blood vessel; and
if the patient has vascular regression then treating the patient with an inhibitor of an Endothelial ETS Family Transcription Factor,
wherein there is a decrease in retinal neovessels or vascular malformations, a decrease in a retinal avascular area, or both a vehicle-injected contralateral eye.

22. The method of claim 21, wherein the inhibitor of an Endothelial ETS Family Transcription Factor is YK 4-279, TK216, or derivatives having the formula, respectively:

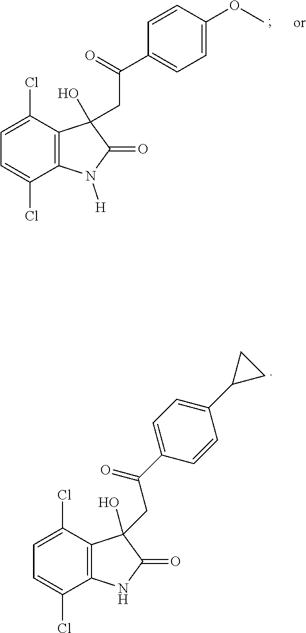 [SMILES: COc1ccc(C(=O)CC2(O)C(=O)Nc3c(Cl)ccc(Cl)c32)cc1 ; O=C(CC1(O)C(=O)Nc2c(Cl)ccc(Cl)c21)c1ccc(C2CC2)cc1]

23. The method of claim 21, wherein the molecule has the formula: 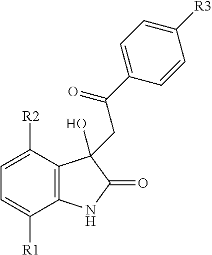
wherein, R1, R2, R3 are the same or different and are each independently hydrogen, halogen, Cl, Br, F, I, OH, a saturated or unsaturated alkyl group, a cycloalkyl group, a substituted or unsubstituted aryl group, an aryl group, an alkoxy group, a nitrone group or a selected from R′COO, R′COOCH(R″), R′CH═CH, R′CONN, R′CONH(R″); wherein R′, R″ are each independently a saturated or unsaturated alkyl, ring, an alkyl group, a substituted or unsubstituted aryl group, or an aromatic hetero group.

24. The method of inducing vascular regression in poorly perfused blood vessels of claim 1, comprising providing the subject with an effective amount of an inhibitor that blocks the interaction between one or more ETS factors and one or more Krüppel-like factor (KLF) proteins,
wherein the subject is in need of treatment for retinopathy of prematurity (ROP), diabetic retinopathy (DR), or vascular malformations, wherein the inhibitor does not inhibit vascular endothelial growth factor (VEGF),
wherein the inhibitor is administered topically, subconjunctivally, intracamerally, subtenonally, subretinally, subchoroidally, suprachoroidally, supraorbitally, retrobulbarlly, as an ocular implant, or intravitreally, and wherein the composition is an eye drop, gel, ointment, spray, a reservoir, or mist; and
wherein the inhibitor at least one of: decrease retinal neovessels or vascular malformations by at least 40% or a retinal avascular area by at least 60% compared to a vehicle-injected contralateral eye.

25. (canceled)

26. (canceled)

27. (canceled)

28. (canceled)
